(19) United States
(12) Patent Application Publication
(10) Pub. No.: US 2022/0143292 A1
(43) Pub. Date: May 12, 2022

(54) TREATING COVID-19 UTILIZING EXTRACORPOREAL RADIOFREQUENCY

(71) Applicant: MARV ENTERPRISES LLC, Hermitage, PA (US)

(72) Inventor: Mitchell S. Felder, Hermitage, PA (US)

(21) Appl. No.: 17/520,171

(22) Filed: Nov. 5, 2021

Related U.S. Application Data
(60) Provisional application No. 63/111,043, filed on Nov. 8, 2020.

(51) Int. Cl. A61M 1/36; A61K 41/00; A61K 39/42

(52) U.S. Cl. CPC A61M 1/3681; A61M 1/369; A61M 1/3687; A61M 2202/0464; A61K 39/42; A61M 2202/206; A61M 2202/0413; A61K 41/0038

(57) ABSTRACT

An embodiment provides a method for treating a body fluid of a patient with Covid-19, including: removing the body fluid from a patient, wherein the body fluid comprises a Covid-19 targeted pathogenic antigen (TPA); applying a treatment to the body fluid, wherein the treatment comprises an antibody that joins with the Covid-19 targeted pathogenic antigen in the body fluid to form an antibody-TPA complex, wherein the antibody comprises a radiofrequency absorption enhancer forming an antibody-radio frequency enhancing moiety-virion complex (ARFVC); removing the ARFVC from the body fluid using a radiofrequency source; and returning the body fluid to the patient. Other aspects are described and claimed.

### CROSS REFERENCE TO RELATED APPLICATION

This application claims priority to U.S. Provisional Patent Application Ser. No. 63/111,043, filed on Nov. 8, 2020, and entitled “METHOD FOR TREATING AND CURING COVID-19 INFECTION BY UTILIZING RADIOFREQUENCY EXTRACORPOREALLY TO ERADICATE THE VIRUS,” the contents of which are incorporated by reference herein.

### FIELD

This application relates generally to a treatment for Covid-19, and, more particularly, to an extracorporeal radiofrequency methodology for the treatment of Covid-19.

### BACKGROUND

Coronaviruses are a family of viruses that can cause illnesses such as severe acute respiratory syndrome (SARS) and Middle East respiratory syndrome (MERS). A new coronavirus (Covid-19) was identified as the cause of a disease outbreak in China. The virus is presently known as the severe acute respiratory syndrome coronavirus 2 (SARS-CoV-2). The disease it causes is called coronavirus disease 2019 (COVID-19).

Cases of COVID-19 have been reported in multiple countries, where it has caused a great deal of morbidity and mortality, in a worldwide pandemic. The disorder is characterized by shortness of breath, increased mucus production, sore throat, cough, and fever. This may necessitate admission to a hospital, with subsequent admission to an intensive care unit for the respiratory support of the infected patient.

There is a need for treatments for Covid-19, such as eradicating the Covid-19 virion from a patient's body fluid, due to the worldwide pandemic of this infection.

### BRIEF SUMMARY

In summary, one embodiment provides a method for treating a body fluid of a patient with Covid-19, comprising: removing the body fluid from a patient, wherein the body fluid comprises a Covid-19 targeted pathogenic antigen (TPA); applying a treatment to the body fluid, wherein the treatment comprises an antibody that joins with the Covid-19 targeted pathogenic antigen in the body fluid to form an antibody-TPA complex, wherein the antibody comprises a radiofrequency absorption enhancer forming an antibody-radio frequency enhancing moiety-virion complex (ARFVC); removing the ARFVC from the body fluid using a radiofrequency source; and returning the body fluid to the patient.

Another embodiment provides a device for treating a body fluid of a patient with Covid-19, comprising: a transparent first stage including an inlet for the body fluid and at least one exterior wall defining a treatment chamber; a transparent second stage, fluidly connected to the first stage, comprising a removal module and an outlet for the body fluid, wherein the treatment chamber comprises a delivery tube for introducing an antibody into the treatment chamber, wherein the delivery tube comprises a hollow tube including at least one interior wall defining a plurality of holes through which the antibody can be added to the treatment chamber, wherein the treatment is delivered through the hollow tube in counter-current mode with reference to the body fluid; and a radiofrequency source; the device being configured to: remove the body fluid from a patient, wherein the body fluid comprises a Covid-19 targeted pathogenic antigen (TPA); apply a treatment to the body fluid, wherein the treatment comprises an antibody that joins with the Covid-19 targeted pathogenic antigen in the body fluid to form an antibody-TPA complex, wherein the antibody comprises a radiofrequency absorption enhancer forming an antibody-radio frequency enhancing moiety-virion complex (ARFVC); remove the ARFVC from the body fluid using a radiofrequency source; and return the body fluid to the patient.

A further embodiment provides a product for treating a body fluid of a patient with Covid-19, comprising: a transparent first stage including an inlet for the body fluid and at least one exterior wall defining a treatment chamber; a transparent second stage, fluidly connected to the first stage, comprising a removal module and an outlet for the body fluid, wherein the treatment chamber comprises a delivery tube for introducing an antibody into the treatment chamber, wherein the delivery tube comprises a hollow tube including at least one interior wall defining a plurality of holes through which the antibody can be added to the treatment chamber, wherein the treatment is delivered through the hollow tube in counter-current mode with reference to the body fluid, wherein the treatment comprises an antibody that joins with a Covid-19 targeted pathogenic antigen (TPA) in the body fluid to form an antibody-TPA complex, wherein the antibody comprises a radiofrequency absorption enhancer forming an antibody-radio frequency enhancing moiety-virion complex (ARFVC); and a radiofrequency source.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates an example partial cross-sectional view of a cylinder and tubing used to deliver a treatment to a bodily fluid.

FIG. 2 illustrates an example a partial cross-sectional view showing additional detail of the cylinder and tubing of FIG. 1.

FIG. 3 illustrates an example series of microscreens for filtering a body fluid.

FIG. 4 illustrates an example flow diagram of a method for treatment of Covid-19 using extracorporeal radiofrequency.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

Covid-19 has spread worldwide and become a global pandemic. The loss of life, suffering, and economic struggles have reached all corners of the globe. Symptoms may manifest about 2-14 days after exposure. The symptoms may include fever, chills, cough, shortness of breath, difficulty breathing, fatigue, muscle/body aches, new loss of taste/smell, sore throat, congestion, runny nose, nausea, vomiting, or diarrhea. More severe symptoms may include trouble breathing, persistent pain/pressure in the chest, confusion, inability to wake or stay awake, or bluish lips/face. Some cases may require hospitalization and even intensive care unit healthcare. Because of the novelty of the virus, very few tests exist that are specific for Covid-19. What is needed is a method for the reduction of Covid-19 transmission.

Coronaviruses are a family of viruses that can cause illnesses such as severe acute respiratory syndrome (SARS) and Middle East respiratory syndrome (MERS). A new coronavirus (Covid-19) was identified as the cause of a disease outbreak in China. The virus is known as the severe acute respiratory syndrome coronavirus 2 (SARS-CoV-2). The disease it causes is called coronavirus disease 2019 (Covid-19).

Accordingly, a method for the extracorporeal treatment of a patient's body fluid, CSF (cerebrospinal fluid) and/or blood, with the utilization of radiofrequency technology for the eradication of the targeted pathogenic antigen(s)/TPA(s) of Covid-19 virus (SARS-CoV-2) that include, but are not limited to, Covid-19 spike glycoprotein, Covid-19 M-Protein, Covid-19 Hemagglutinin-esterase dimer, Covid-19 Envelope, Covid-19 E-Protein, Covid-19 N-Protein, nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex, wherein the antibody comprises a tag sensitive to a radiofrequency; in said body fluid is described herein. Disclosed is a method for the extracorporeal treatment of a patient's body fluid with the utilization of radiofrequency for the eradication of targeted pathogenic antigen(s)/TPA(s) of Covid-19 virus (SARS-CoV-2) in the body fluid. Bodily fluid can be selected from a group consisting of cerebrospinal fluid (CSF) and blood, and combinations thereof. The treatment includes stages comprising removing the body fluid from a patient which contains Covid-19 pathogenic antigens, exposing and binding those target pathogenic antigen(s) to antibodies forming TA-antibody complexes, eradicating the antibody bound Targeted Pathogenic Antigen(s)/TPAs utilizing radiofrequency (RF), and then returning the body fluid to the patient.

The illustrated example embodiments will be best understood by reference to the figures. The following description is intended only by way of example, and simply illustrates certain example embodiments.

In an embodiment, a method or device for the extracorporeal treatment of a patient's body fluid with the utilization of the Kanzius methodology for the eradication of the Covid-19 virion in the body fluid is described. The bodily fluid may be selected from a group consisting of blood, cerebrospinal fluid (CSF), and combinations thereof. The treatment may include stages which may include removing the body fluid from a patient which contains Covid-19 virions, exposing and binding those virions to antibodies forming virion antibody complexes, eradicating the antibody bound virions utilizing the radiofrequency (RF) provided by the Kanzius methodology, and then returning the body fluid to the patient.

In an embodiment, method may treat a patient's body fluid extracorporeally with conjugated antibody(s) designed to react with and bind up particular targeted pathogenic antigen(s)/TPA(s) of Covid-19 virus (SARS-CoV-2) that include, but are not limited to, Covid-19 spike glycoprotein, Covid-19 M-Protein, Covid-19 Hemagglutinin-esterase dimer, Covid-19 Envelope, Covid-19 E-Protein, Covid-19 N-Protein, nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex.

Short-duration emissive energy from a high energy radiation emissive source may then be used to eradicate the virion antibody complexes in the body fluid, and then returning the body fluid back to the patient.

In an embodiment, in a first stage, a body fluid is withdrawn from a patient using standard medical techniques. A convenient method for removing blood is by using the standard venipuncture technique. A convenient method for removing the CSF is by using the standard lumbar puncture technique. Other techniques known to those skilled in the art are contemplated by this disclosure.

In an embodiment, in a second stage a treatment is applied to a body fluid extracorporeally. The treatment includes exposing the body fluid to a tagged antibody generated to bind specific targeted pathogenic antigens (TPAs) of the Covid-19 virus such as those described above. During this treatment the conjugated antibody(s) and the targeted pathogen antigen form virion antibody complexes.

In an embodiment, a method for enhancing radiofrequency (RF) absorption may include providing targeted RF enhancers, such as antibodies with an attached RF absorption enhancer, such as, for example metal particles. The antibodies target and bind to the Covid-19 virion. Binding RF enhancing particles to the antibodies (and other carriers having at least one targeting moiety) permits the injection of the antibodies (and other carriers having at least one targeting moiety) into the extracorporeal target solution.

The RF enhancers may induce the absorption of energy in the antibody-RF enhancing moiety virion complex (ARFVC). In addition, a combination of antibodies (and other carriers having at least one targeting moiety) bound to different metals (and other RF absorbing particles) can be used allowing for variations in the RF absorption characteristics in the extracorporeal target area.

In an embodiment, the energy of the emitted radio frequency (RF) annihilates the antibody-RF enhancing moiety-virion complex (ARFVC), thereby destroying its disease-causing potential. The entire system may be monitored and controlled utilizing a computer, in real time, utilizing time units of 1 millisecond or less during the entire procedure. Persons having ordinary skill in art will recognize that the steps described above can be performed on various devices, machines, or systems. This disclosure contemplates all known devices, machines, or systems that can perform the steps described in the above illustrative example.

In an embodiment, the second stage substantially eliminates, through a high-energy radiofrequency emissive source targeting and annihilating, the antibody-RF enhancing moiety-virion complex (ARFVC) in the body fluid.

In an embodiment, a method for killing the Covid-19 virus may be performed by introducing into the extracorporeal patient body fluid (blood or CSF) RF absorption enhancers capable of selectively binding to the target virions and further capable of generating sufficient heat to kill or damage the bound target antibody-virion complexes by heat generated solely by the application of an RF field generated by an RF signal between a transmission head and a reception head.

In an embodiment, to achieve the eradication of the Covid-19 virions, one may arrange for the transmission and reception heads on opposite sides of a portion of the extra-corporeal body fluid sample for treatment. Irradiating the portion of the extra-corporeal body fluid sample between the transmission and reception heads containing RF absorption enhancers with an RF field kills or damages the target Covid-19 virions/SARS-CoV-2 from the heat generated by the RF absorption enhancers.

The body fluid, from which the target Covid-19 virions/SARS-CoV-2 have been eradicated, may then be returned to the patient, free of the infectious virions. Persons having ordinary skill in art will recognize that the steps described above can be performed on various devices, machines, or systems. This disclosure contemplates all known devices, machines, or systems that can perform the steps described in the above illustrative example.

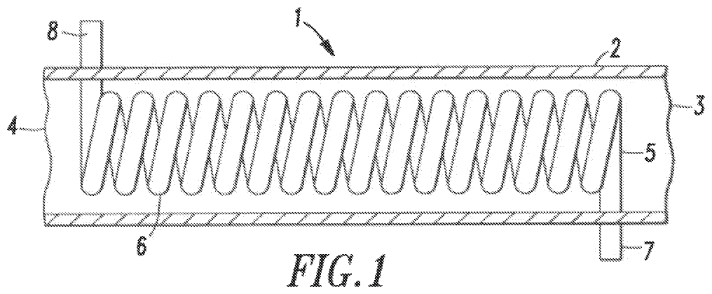

Referring to FIG. 1, in an embodiment, the antibody-RF enhancing moiety, targeting the targeted pathogenic antigen (TPA, such as Covid-19 virus) can be delivered in a concurrent or counter-current mode with reference to the flow of the body fluid. An example of a device that can be utilized in the disclosed method is shown in FIG. 1. The device may include an exterior wall 2 to surround a treatment chamber 5. The treatment of the body fluid conveniently can be applied in the treatment chamber 5. Residence times of the body fluid in the device can be altered by changing the dimensions of the treatment chamber, or by using a dialysis vacuum pump. With reference to FIG. 1, body fluid enters the inlet 3, passes through the treatment chamber 5, and exits the outlet 4. The treatment of an antibody with an attached tagged moiety or targeting the TPA: Covid-19 virus can be applied from a delivery tube 6 located within the treatment chamber 5. Again, the Abs (antibodies), targeting the Covid-19 virus TPA(s) can be delivered in a concurrent or counter-current mode with reference to the flow of the body fluid. In counter-current mode, the body fluid enters the treatment chamber 5 at the inlet 3. This can enter through a first lead 8 near the outlet 4 of the treatment chamber 5. Body fluid then passes to the outlet 4 and the Ab-TPAs pass to the second lead 7 near the inlet 3.

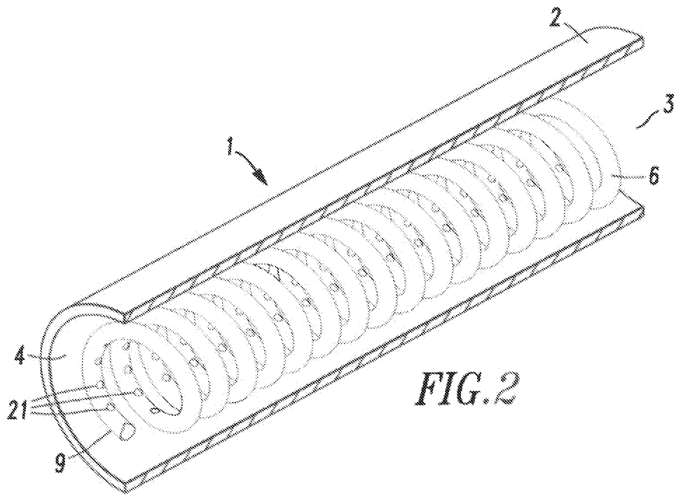

Referring to FIG. 2, in an embodiment, the delivery tube 6 can be hollow and with a plurality of holes 21. The antibody-RF enhancing moiety-virion complexes (ARFVC) can be pumped through the delivery tube 6 to achieve a desired concentration of antibody-RF enhancing moiety-virion complex (ARFVC) in the body fluid. The moiety-virion complexes (ARFVC) perfuse through the holes 21. An inferior wall 9 defines the delivery tube 6. The delivery tube 6 can include at least one lead 7, 8. The lead 7, 8 can deliver the treatment to the treatment chamber 5. Conveniently, the delivery tubes 6 will have a high contact surface area with the body fluid. As shown, the delivery tube 6 comprises a helical coil. The delivery tube 6 can include any suitable material including, for example, metal, plastic, ceramic, or combinations thereof. The delivery tube 6 can also be rigid or flexible. In one embodiment, the delivery tube 6 is a metal tube perforated with a plurality of holes. Alternatively, the delivery tube 6 can be plastic. Example illustrations shown in FIG. 1 and FIG. 2 are non-limiting examples of a device that can be used in the described method and are used for illustrative purposes only.

In an embodiment, as an alternative to using a type of device as described above, the Covid-19 virions are captured using antibody microarrays containing antibody-RF enhancing moieties in microarrays. An antibody microarray is a protein microarray; a collection of capture antibodies is fixed on a solid surface, such as glass, plastic, and silicon chip for the purpose of detecting antigens. Antibody microarrays are composed of millions of identical monoclonal antibodies attached at high density on glass or plastic slides, all of which are transparent. Any microarrays known by those skilled in the art sufficient to perform the described technique are contemplated by this disclosure.

In an embodiment, there may be an extracorporeal exposure of the Targeted Antigens/TAs (SARS-CoV-2). This may create an antibody pathogen complex on the microarray. The complexes are then tracked using an appropriate sensor and obliterated using a high energy focused emissive energy by the methodology of Kanzius. Steps in the process may be monitored and controlled by a computer in real time. Persons having ordinary skill in art will recognize that the steps described above can be performed on various devices, machines, or systems. This disclosure contemplates all known devices, machines, or systems that can perform the steps described in the above illustrative example.

In an embodiment, to eradicate the virion-antibody complexes after exposure in the microarrays, the body fluid may then be forced through a container constructed from a transparent material such as glass, or other material, which exposes the antibody-RF enhancing moiety-virion complex (ARFVC) to a sensing device. The sensing device also creates an enlarged, magnified visual image of the antibody-RF enhancing moiety-virion complex (ARFVC). A concentrated and focused intense energy beam, such as light, may be used to properly illuminate and would use optical pattern recognition of the antibody-RF enhancing moiety-virion complex (ARFVC). Persons having ordinary skill in art will recognize that the steps described above can be performed on various devices, machines, or systems. This disclosure contemplates all known devices, machines, or systems that can perform the steps described in the above illustrative example.

The temperature of the treated body fluid may be maintained at 98.6° F. via continuous cooling of the body fluid using a standard cooling apparatus. A constant thermostatic measurement and control system continuously monitors the process to maintain the body fluid temperature at 98.6° F. Persons having ordinary skill in art will recognize that the steps described above can be performed on various devices, machines, or systems. This disclosure contemplates all known devices, machines, or systems that can perform the steps described in the above illustrative example.

In an embodiment, Covid-19 virion target antigen(s)/TA(s): Covid-19 spike glycoprotein, Covid-19 M-Protein, Covid-19 Hemagglutinin-esterase dimer, Covid-19 Envelope, Covid-19 E-Protein, Covid-19 N-Protein, nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex may be identified and differentiated using standard ELISA methodology. Identification can be done before treatment to determine which TAs, and their concentrations, are present in a patient's CSF and/or blood, and after treatment to analyze the efficiency of removal of the TA. ELISA (enzyme-linked immunosorbent assay) is a biochemical technique which allows for the detection of an antigen in a sample. In ELISA an antigen may be affixed to a surface, and then an antibody is utilized for binding to the antigen. The antibody is linked to an enzyme which enables a color change in the substrate. Other strategies may be employed to validate the level of target antigen(s)/TA(s) in the body fluid before or after treatment: Western blotting technology, UV/Vis spectroscopy, mass spectrometry, and surface plasmon resonance (SPR). Another alternative methodology would utilize a molecular weight cut-off filtration. Molecular weight cut-off filtration refers to the molecular weight at which at least 80% of the target antigen(s)/TA(s) is prohibited from membrane diffusion.

In an embodiment, a portion of the purified body fluid may be tested to ensure that an acceptable portion of the Covid-19 virus targeted antigen(s)/TA(s) has been successfully removed from the body fluid using methods discussed throughout this application. Testing can determine the length of treatment and evaluate the efficacy of the eradication methodology in removing the targeted antigens. Body fluid with an unacceptably high concentration of virion-antibody complexes remaining can then be re-treated before returning the body fluid to the patient.

The treatment may eradicate the targeted Covid-19 virus antigen(s) and subsequently the virus particles from the body fluid. The cleansed body fluid may then be returned to the patient, for example by using the same catheter that was originally used in removing the body fluid. In one non-limiting embodiment, the treatment of blood includes removing 20 ml to 500 ml (milliliters) of blood from a patient, and then applying the treatment to the blood before returning it to the patient. The frequency of such treatments would depend upon an analysis of the underlying symptomatology and pathology of the patient.

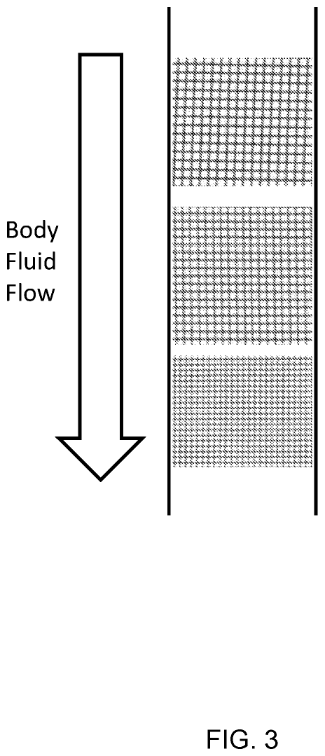

Referring to FIG. 3, in an embodiment, a methodology of the present intervention, which may separate the TA (target antigen(s) from re-entering the body fluid (CSF or blood) of the patient being treated may use a designer antibody with an attached macromolecular moiety, such as a metallic moiety. The macromolecular moiety, attached to the antibody, would be 1.000 mm to 0.0000001 mm in diameter. The antibody-macromolecular moiety-targeted antigen complex would then be blocked from reentering the patient's blood, using a series of microscreens which contain openings with a diameter 50% to 99.99999% less than the diameter of the designer antibody-macromolecular moiety (See FIG. 3). The microscreen opening(s) may need to have a diameter of at least 25 μm to permit the passage and return to the circulation of the non-pathological blood constituents. However, the screen opening(s) may have much greater apertures when specifically dealing with CSF or blood plasma not containing blood elements such as white blood cells, red blood cells, platelets, etc. utilizing plasmapheresis techniques.

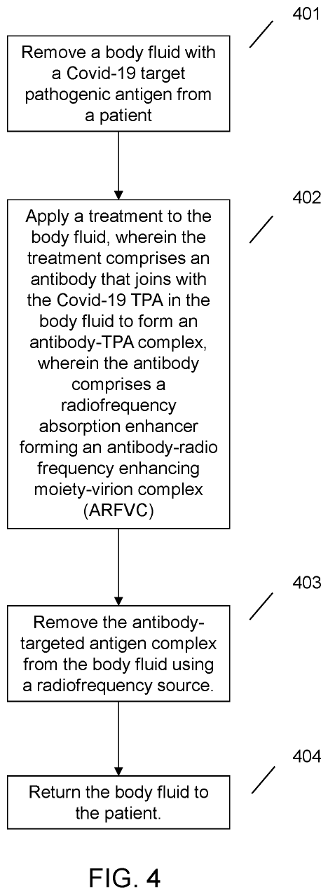

Referring to FIG. 4, an example method is illustrated. A method for treating a body fluid comprising: a first stage including removing the body fluid from a patient at 401; a second stage including applying a treatment to the body fluid, wherein the treatment comprises an antibody that joins with the Covid-19 targeted pathogenic antigen in the body fluid to form an antibody-TPA complex, wherein the antibody comprises a radiofrequency absorption enhancer forming an antibody-radio frequency enhancing moiety-virion complex (ARFVC), wherein the antibody comprises a radiofrequency enhancer at 402, and removing the antibody-RF enhancing moiety-virion complex from the body fluid with a radiofrequency source at 403; and a third stage including returning the body fluid to the patient at 404.

Unless otherwise indicated, all numbers expressing quantities of ingredients, properties such as molecular weight, reaction conditions, and so forth used in the specification and claims are to be understood as being modified in all instances by the term “about.”

Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

While the invention has been particularly shown and described with reference to a preferred embodiment, it will be understood by those skilled in the art that various changes in form and detail may be made therein without departing from the spirit and scope of the invention.

Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

It can be appreciated from the foregoing that electronic components of one or more systems or devices may include, but are not limited to, at least one processing unit, a memory, and a communication bus or communication means that couples various components including the memory to the processing unit(s). A system or device may include or have access to a variety of device readable media. System memory may include device readable storage media in the form of volatile and/or nonvolatile memory such as read only memory (ROM) and/or random access memory (RAM). By way of example, and not limitation, system memory may also include an operating system, application programs, other program modules, and program data.

Embodiments may be implemented as an instrument, system, method or program product. Accordingly, an embodiment may take the form of an entirely hardware embodiment, or an embodiment including software (including firmware, resident software, micro-code, etc.) that may all generally be referred to herein as a “circuit,” “module” or “system.” Furthermore, embodiments may take the form of a program product embodied in at least one device readable medium having device readable program code embodied thereon.

A combination of device readable storage medium(s) may be utilized. In the context of this document, a device readable storage medium (“storage medium”) may be any tangible, non-signal medium that can contain or store a program comprised of program code configured for use by or in connection with an instruction execution system, apparatus, or device. For the purpose of this disclosure, a storage medium or device is to be construed as non-transitory, i.e., not inclusive of signals or propagating media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and products according to various example embodiments. It will be understood that the actions and functionality may be implemented at least in part by program instructions. These program instructions may be provided to a processor of a device, e.g., a hand-held measurement device, or other programmable data processing device to produce a machine, such that the instructions, which execute via a processor of the device, implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method for treating a body fluid of a patient with Covid-19, comprising:
removing the body fluid from a patient, wherein the body fluid comprises a Covid-19 targeted pathogenic antigen (TPA);
applying a treatment to the body fluid, wherein the treatment comprises an antibody that joins with the Covid-19 targeted pathogenic antigen in the body fluid to form an antibody-TPA complex, wherein the antibody comprises a radiofrequency absorption enhancer forming an antibody-radio frequency enhancing moiety-virion complex (ARFVC);
removing the ARFVC from the body fluid using a radiofrequency source; and
returning the body fluid to the patient.

2. The method of claim 1, wherein the Covid-19 targeted pathogenic antigen is selected from the group consisting of: Covid-19 spike glycoprotein, Covid-19 M-Protein, Covid-19 Hemagglutinin-esterase dimer, Covid-19 Envelope, Covid-19 E-Protein, Covid-19 N-Protein, nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex.

3. The method of claim 1, wherein the radiofrequency absorption enhancer comprises a metal.

4. The method of claim 1, wherein the radiofrequency source comprises a short-duration emissive energy from a high energy radiation emissive source.

5. The method of claim 1, wherein the radiofrequency source annihilates the antibody-radio frequency enhancing moiety-virion complex (ARFVC) and a disease-causing potential of the Covid-19 targeted pathogenic antigen.

6. The method of claim 1, wherein the radiofrequency source generates heat to damage the bound Covid-19 targeted pathogenic antigen by heat generated solely by the application of a radiofrequency field generated by a radiofrequency signal between a transmission head and a reception head.

7. The method of claim 1, further comprising capturing the antibody-radio frequency enhancing moiety-virion complex (ARFVC) using an antibody microarray.

8. The method of claim 1, further comprising determining an efficacy of treatment, using the radiofrequency source, based on testing the body fluid after the treating the body fluid and before returning the body fluid to the patient.

9. The method of claim 1, wherein the removal further comprises a sensor for detecting the antibody-radio frequency enhancing moiety-virion complex (ARFVC).

10. The method of claim 1, wherein the body fluid is selected from the group consisting of: cerebrospinal fluid (CSF) and blood.

11. A device for treating a body fluid of a patient with Covid-19, comprising:
a transparent first stage including an inlet for the body fluid and at least one exterior wall defining a treatment chamber;
a transparent second stage, fluidly connected to the first stage, comprising a removal module and an outlet for the body fluid, wherein the treatment chamber comprises a delivery tube for introducing an antibody into the treatment chamber, wherein the delivery tube comprises a hollow tube including at least one interior wall defining a plurality of holes through which the antibody can be added to the treatment chamber, wherein the treatment is delivered through the hollow tube in counter-current mode with reference to the body fluid; and
a radiofrequency source;
the device being configured to:
remove the body fluid from a patient, wherein the body fluid comprises a Covid-19 targeted pathogenic antigen (TPA);
apply a treatment to the body fluid, wherein the treatment comprises an antibody that joins with the Covid-19 targeted pathogenic antigen in the body fluid to form an antibody-TPA complex, wherein the antibody comprises a radiofrequency absorption enhancer forming an antibody-radio frequency enhancing moiety-virion complex (ARFVC);
remove the ARFVC from the body fluid using a radiofrequency source; and
return the body fluid to the patient.

12. The device of claim 11, wherein the Covid-19 targeted pathogenic antigen is selected from the group consisting of: Covid-19 spike glycoprotein, Covid-19 M-Protein, Covid-19 Hemagglutinin-esterase dimer, Covid-19 Envelope, Covid-19 E-Protein, Covid-19 N-Protein, nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex.

13. The device of claim 11, wherein the radiofrequency absorption enhancer comprises a metal.

14. The device of claim 11, wherein the radiofrequency source comprises a short-duration emissive energy from a high energy radiation emissive source.

15. The device of claim 11, wherein the radiofrequency source annihilates the antibody-radio frequency enhancing moiety-virion complex (ARFVC) and a disease-causing potential of the Covid-19 targeted pathogenic antigen.

16. The device of claim 11, wherein the radiofrequency source generates heat to damage the bound Covid-19 targeted pathogenic antigen by heat generated solely by the application of a radiofrequency field generated by a radiofrequency signal between a transmission head and a reception head.

17. The device of claim 11, further comprising capturing the antibody-radio frequency enhancing moiety-virion complex (ARFVC) using an antibody microarray.

18. The device of claim 11, further comprising determining an efficacy of treatment, using the radiofrequency source, based on testing the body fluid after the treating the body fluid and before returning the body fluid to the patient.

19. The device of claim 11, wherein the removal further comprises a sensor for detecting the antibody-radio frequency enhancing moiety-virion complex (ARFVC).

20. A product for treating a body fluid of a patient with Covid-19, comprising:
a transparent first stage including an inlet for the body fluid and at least one exterior wall defining a treatment chamber;
a transparent second stage, fluidly connected to the first stage, comprising a removal module and an outlet for the body fluid, wherein the treatment chamber comprises a delivery tube for introducing an antibody into the treatment chamber, wherein the delivery tube comprises a hollow tube including at least one interior wall defining a plurality of holes through which the antibody can be added to the treatment chamber, wherein the treatment is delivered through the hollow tube in counter-current mode with reference to the body fluid, wherein the treatment comprises an antibody that joins with a Covid-19 targeted pathogenic antigen (TPA) in the body fluid to form an antibody-TPA complex, wherein the antibody comprises a radiofrequency absorption enhancer forming an antibody-radio frequency enhancing moiety-virion complex (ARFVC); and
a radiofrequency source.
